(19) United States
(12) Patent Application Publication
(10) Pub. No.: US 2007/0071780 A1
(43) Pub. Date: Mar. 29, 2007

(54) Personal care composition comprising a perfume booster accord

(76) Inventors: Zerlina Guzdar Dubois, Mason, OH (US); Jose Maria Velazquez, Berkshire (GB); Michael Frederick Niebauer, Cincinnati, OH (US); Robert Richard Dykstra, West Chester, OH (US); Virginia Tzung-Hwei Hutchins, Cincinnati, OH (US); George Endel Deckner, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/453,599

(22) Filed: Jun. 15, 2006

Related U.S. Application Data
(60) Provisional application No. 60/691,176, filed on Jun. 16, 2005.
(60) Provisional application No. 60/694,756, filed on Jun. 28, 2005.

(51) Int. Cl. A61K 8/02; A61K 8/49; A61K 8/37; A61K 8/36

(52) U.S. Cl. 424/401; 512/1

(57) ABSTRACT

A personal care composition comprising a perfume booster accord. The human nose can detect the odor of the perfume booster accord at low concentrations. The perfume booster accord also provides a bigger bloom and longevity of scent. The present invention is further directed to a method of using the personal care composition.

### CROSS REFERENCE TO RELATED APPLICATIONS

This application claims the benefit of U.S. Provisional Application Ser. No. 60/691,176, filed Jun. 16, 2005, and U.S. Provisional Application Ser. No. 60/694,756, filed Jun. 28, 2005.

### FIELD

The present invention relates to personal care compositions comprising a perfume booster accord. The human nose can detect the odor of the perfume booster accord at low concentrations. The perfume booster accord also provides a bigger bloom and longevity of scent. These compositions are suitable for use on mammalian skin and hair.

### BACKGROUND

Typical perfumes intended for skin and hair compositions mainly focus on delivering the most pleasant character for the application. However, these perfumes do not fully optimize their blooming, longevity, malodor coverage, etc., and they can be costly. Moreover, consumers and new technologies are demanding more performing perfumes. Therefore, there is a need to create perfume booster accords to improve targeted aspects of a given perfume creation while maintaining their hedonistic aspect.

### SUMMARY

The present invention is directed to personal care compositions comprising a perfume booster accord. In one exemplary embodiment, the booster accord is present in an amount of from about 0.01% to about 10% by weight of the personal care composition. The perfume booster accord comprises at least two high odor value materials having an odor detection threshold of less than or equal to about 50 ppb and a human recognition slope factor greater than or equal to about 600. The high odor value materials are present in an amount of from about 0.05% to about 100% by weight of the perfume booster accord.

The present invention is further directed to methods for selecting high odor value materials for use in perfume composition (e.g., a perfume booster accord composition). In one exemplary embodiment, the method comprises the steps of: (a) identifying a first list of high odor value materials; (b) determining an odor detection threshold value of each of said high odor value materials; (c) determining a human recognition slope factor value of each of said high odor value materials; and (d) selecting said high odor value materials that have both an odor detection threshold value that is less than or equal to about 50 ppb, and a human recognition slope factor value that is greater than or equal to about 600.

### DETAILED DESCRIPTION

The present invention relates to personal care compositions comprising at least one perfume booster accord. It has been found that the perfume booster accords of the present invention are detectable by the human nose at low concentrations. The perfume booster accord of the present invention also provides a bigger bloom and longevity of scent. The compositions of the present invention are suitable for use on mammalian skin and hair.

The essential components of the personal care composition are described below. Also included is a nonexclusive description of various optional and preferred components useful in embodiments of the present invention. While the specification concludes with claims that particularly point out and distinctly claim the invention, it is believed the present invention will be better understood from the following description.

All percentages, parts, and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

The compositions and methods/processes of the present invention can comprise, consist of, and consist essentially of the essential elements and limitations of the invention described herein, as well as any of the additional or optional ingredients, components, steps, or limitations described herein.

By the term “visually distinct,” as used herein, is meant that the regions occupied by each phase can be separately seen by the human eye as distinctly separate regions in contact with one another (i.e., they are not emulsions or dispensions of particles of less than about 100 microns). In one preferred embodiment of the present invention, the cleansing phase and the benefit phase are present within the container as distinct layers or stripes. The stripes may be relatively uniform and even across the dimensions of the package. Alternatively, the layers may be uneven, i.e., wavy, or may be nonuniform in dimension. The stripes do not need to necessarily extend across the entire dimension of the package. The stripe can be various geometric shapes, various colors, or include glitter or pearlescence.

By the term “visibly clear,” as used herein, the transparency of the composition is measured using Ultra-Violet/Visible (UV/VIS) Spectrophotometry, which determines the absorption or transmission of UV/VIS light by a sample. A light wavelength of 600 nm has been shown to be adequate for characterizing the degree of clarity of cosmetic compositions. Typically, it is best to follow the specific instructions relating the specific spectrophotometer being used. In general, the procedure for measuring percent transmittance starts by setting the spectrophotometer to the 600 nm. Then a calibration “blank” is run to calibrate the readout to 100 percent transmittance. The test sample is then placed in a cuvette designed to fit the specific spectrophotomer and the percent transmittance is measured by the spectrophotomer at 600 nm. By the term “multi-phased” or “multi-phase,” as used herein, is meant that at least two phases occupy separate and distinct physical spaces inside the package in which they are stored, but are in direct contact with one another (i.e., they are not separated by a barrier and they are not emulsified or mixed to any significant degree). In one preferred embodiment of the present invention, the “multi-phased” personal care compositions comprising at least two phases are present within the container as a visually distinct pattern. The pattern results from the mixing or homogenization of the “multi-phased” composition. The patterns include but are not limited to the following examples: striped, marbled, rectilinear, interrupted striped, checked, mottled, veined, clustered, speckled, geometric, spotted, ribbons, helical, swirled, arrayed, variegated, textured, grooved, ridged, waved, sinusoidal, spiraled, twisted, curved, cycle, streaked, striated, contoured, anisotropic, laced, weave or woven, basket weave, spotted, and tessellated. Preferably the pattern is selected from the group consisting of striped, geometric, marbled and combinations thereof. In a preferred embodiment, the striped pattern may be relatively uniform and even across the dimension of the package. Alternatively, the striped pattern may be uneven, i.e., wavy, or may be non-uniform in dimension. The striped pattern does not need to necessarily extend across the entire dimension of the package. The phases may be various different colors, or include particles, glitter or pearlescence.

The term “stable,” as used herein, unless otherwise specified, refers to compositions that maintain at least two “separate” phases when sitting in physical contact at ambient conditions for a period of at least about 180 days wherein the distribution of the two phases in different locations in the package does not visibly change over time. By “separate” it is meant that the well-distributed nature of the visually distinct phases is compromised, such that larger regions of at least one phase collect until the balanced dispensed ratio of the two or more compositions relative to each other is compromised.

The term “personal care composition,” as used herein, unless otherwise specified, refers to the compositions of the present invention, wherein the compositions are intended to include only those compositions for topical application to the hair or skin, and specifically excludes those compositions that are directed primarily to other applications such as hard surface cleansing, fabric or laundry cleansing, and similar other applications not intended primarily for topical application to the hair or skin. Personal care compositions may include, but are not limited to: shampoo, conditioner, antiperspirant, deodorant, hair styling products, cleansers, soaps, bar soap, bodywash, cosmetics, foundations, lotions, creams, ointments, and hydro-alcoholic solutions.

It is to be understood that when a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

A. Perfume Booster Accord

The personal care compositions of the present invention comprise a perfume booster accord that is suitable for application to the hair or skin. The perfume booster accord is present in an amount of from about 0.01% to about 10% of the personal care composition, preferably from about 0.05% to about 5% of the personal care composition, more preferably from about 0.1% to about 1% of the personal care composition.

A perfume booster accord is a perfume composition that comprises at least two high odor value materials. The high odor value materials are components of perfume booster accords that improve the perceived odor intensity of a traditional fragrance. Alternatively, the high odor value materials and perfume booster accords minimize the required mass of fragrance while maintaining the intended odor intensity.

Preferably the perfume booster accord comprises at least five high odor value materials, including, for example: 5, 6, 7, 8, 9, 10, 11, 12, 13, 14, 15, 16, 17, 18, 19, 20, 21, 22, 23, 24, 25, 26, 27, 28, 29, or 30 high odor value materials, more preferably at least ten high odor value materials, including, for example, 10, 11, 12, 13, 14, 15, 16, 17, 18, 19, or 20 odor value materials. In some embodiments, the perfume booster accord comprises at least twenty high odor value materials. The high odor value materials of the present invention are present in an amount of from about 0.05% to about 100% of the perfume booster accord, more preferably from about 0.10% to about 70% of the perfume booster accord.

The high odor value materials of the present invention are defined as materials that have an odor detection threshold of less than or equal to about 50 ppb, preferably less than or equal to about 25 ppb, more preferably less than or equal to about 10 ppb.

The high odor value materials of the present invention may have a human recognition slope factor greater than or equal to about 600, preferably greater than or equal to about 800.

Odor detection thresholds are determined using a gas chromatograph. The gas chromatograph is calibrated to determine the exact volume of material injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of material.

For example, to determine whether a material has a threshold below 50 ppb, solutions are delivered to the sniff port at the back-calculated concentration. A panelist sniffs the GC effluent and identifies the retention time when odor is noticed. The average across all panelists determines the threshold of noticeability. The necessary amount of analyte is injected onto the column to achieve a 50 ppb concentration at the detector. Typical gas chromatograph parameters for determining odor detection thresholds are listed below.

GC: 5890 Series II with FID detector

7673 Autosampler

Column: J&W Scientific DB-1

Length 30 meters ID 0.25 mm film thickness 1 micron

Method:

Split Injection: 17/1 split ratio

Autosampler: 1.13 microliters per injection

Column Flow: 1.10 ml/minute

Air Flow: 345 ml/minute

Inlet Temp. 245° C.

Detector Temp. 285° C.

Temperature Information

Initial Temperature: 50° C.

Rate: 5° C./minute

Final Temperature: 280° C.

Final Time: 6 minutes

Leading assumptions:

(i) 12 seconds per sniff

(ii) GC air adds to sample dilution

Examples of materials that have odor detection thresholds as described in the present invention include, but are not limited to, those listed selected from the group consisting of cyclemax, ethyl 2 methyl butyrate, ethyl 2,4 decadienoate, allyl heptoate, amyl acetate, ethyl butyrate, Grapefruit Zest (C&A), prenyl acetate, pinoacetaldehyde, maltol, ethyl maltol, 2,6 nonadienal, 2,6 nonadienol, 3,6 nonadienol, cis-6 nonenol, excital, ebanol, polysantol, orange juice carbonyls, lemon juice carbonyls, orange sinensal, paradiff, tangerinal, benzaldehyde, mandarin aldehyde, undecalactone, nonalactone, gamma decalactone, norlimbanol, vanillin, ethyl vanillin, decyl aldehyde, undecylenic aldehyde, trans-2-hexenal, trans-2-decenal, damascenone, 2-isobutylthiazole, 4-methyl-4-mercaptopentan-2-one, corps cassis 0.1% TEC, patchouli, 2-methoxy-4-vinylphenol, pyridine acetyl 10%, sulfurol, diacetyl, furaneol, maple lactone, allyl amyl glycolate, Ambroxan, alpha damascone damascene, Cetalox, cyclal C, Cedramber, cyclo galbanate, Galbex, Cymal, nerol, Florhydral, P.t. bucinal, iso cyclo citral, Fructone, methyl iso butenyl tetrahydro pyran, Frutene, Delphone, ethyl methyl phenyl glycidate, Violiff, flor acetate, Delta damascone damascene, Ambrox, Calone, iso eugenol, Hivemal, methyl beta napthyl ketone, Ozonil, benzyl salicylate, Spirogalbone, cinnamic alcohol, Javanol, dihydro iso jasmonate, Adoxal, Kharismal, pyrazines, ethyl anthranilate, aldehyde supra, Bacdanol, Anethol, irisantheme, yara yara, Keone, cis 3 hexenyl salicylate, methyl nonyl ketone, coumarin, gamma dodecalactone, eugenol, Applinate, eucalyptol, intreleven aldehyde, heliotropin, Triplal, indol, Manzanate, ionone, alpha, trans 4 decenal, ionone beta, Oxane, linalool, neobutanone, methyl anthranilate, Clonal, methyl octine carbonate, Floralozone, methyl heptine carbonate, Sandalore, methyl nonyl acetaldehyde, Cashmeran, para hydroxy phenyl butanone, phenoxy ethyl iso butyrate, phenyl acetaldehyde, ethyl methyl phenyl glycidate, undecyl aldehyde, Aurantiol, nectaryl, iso e super, melonal, Lyral, buccoxime, Lauric aldehyde, nirvanol, Trifemal, pyrazobutyle, Veloutone, undecavertol, Anisic aldehyde, paramenthene, isovaleric aldehyde 0.1% DPG, liminal, labienoxime, rhubofix, and isopropyl quinoline.

The Human Recognition Slope Factor (HRSF) is a tool in choosing the most efficient high odor value materials to create perfume booster accords. It is a number that shows how efficient a high odor value material is in creating different odor intensity impressions on humans as a function of its mass when applied to a composition. The higher the number, the more efficient the material will be.

The HRSF identifies the behavior of perfume materials through the relevant range of concentrations in a given product. The calculation process of the HRSF also defines the concentration where the high odor value material reaches its maximum intensity in a given media.

The HRSF is determined by the following calculations:
HRSF=Ii/xi
wherein:
HRSF=Human Recognition Slope Factor;
Ii is the maximum intensity that a high odor value material delivers in a given media; and
xi is the lowest concentration of the high odor value material to reach maximum intensity.

The value of Ii and xi are calculated using the following two equations. The first equation (I) describes the intensity of a high odor value material across a broad range of concentrations. The second equation (VI) allows one to find the maximum concentration when both mathematical equations reach the same intensity value.
I=a/(1+(x/c)ˆb) (I)
wherein:
I is the Intensity of the high odor value material;
x is the concentration of the material; and
a, b, and c are experimental constants for each high odor value material.

The experimental constants of “a, b, and c” are generated by performing a non linear regression analysis using commercially available software. One software package useful in performing the non linear regression analysis is GraphPad Prism 4 for Windows, available from GraphPad Software, Inc., San Diego Calif.

Odor evaluations of each raw material are conducted among at least 25 people. Each material is diluted to a given concentration, and panelists are requested to grade the smell using a numeric scale (Low: 0-15, Slight: 16-30, Moderate: 31-50, Strong: 51-70, Very Strong: 71-up). Internal controls containing Benzyl salicylate, Citronellol and Ethyl 2 Methyl Butyrate are used to define the standard intensity for each range.

An illustration of how the non linear regression analysis works is described below. In the following example, the material Alpha Damascone was used for illustrative purposes. The experimental data from 25 panelists indicate that the average value for each observation of Alpha Damascone in DPG is as follows:

| Concentration (X) | Odor Intensity (I) |
|---|---|
| 0.0004 | 5 |
| 0.0022 | 15 |
| 0.0032 | 25 |
| 0.0044 | 35 |
| 0.0072 | 50 |
| 0.0092 | 55 |
| 0.0170 | 60 |

The goal of the software package is to minimize the sum of the squares of the vertical distances of the points from the curve. The software prompts the user to perform the following steps to run an iterative process that allows one to find the values of a, b, and c. The R² factor (shown below) will indicate if the objective of minimizing the sum of the squares of the vertical distances of the points from the curve was achieved. The closer the R² factor is to 1.0, the better the “best-fit values” will be.

Step 1: Enter the “X” and “I” data in the data table provided by the software.

Step 2: Enter the equation being used: I=a/(I+(x/c)ˆb)

Step 3: Enter the following Initial Values when prompted:

a=50

b=−1.0

c=0.001

While the numbers listed above will provide “a, b, and c” values, the range of initial values which will also produce the same or substantially the same “a, b, and c” values is as follows:

a: 30-60

b: 0-(−5)

c: 0-0.1

The software generates the curve defined by the initial values, calculates the sum-of-squares (the sum of the squares of the vertical distances of the points from the curve), adjusts the variables to make the curve come closer to the data points, adjusts the variables again so that the curve comes even closer to the points, keeps adjusting the variables until the adjustments make virtually no difference in the sum-of-squares and reports the best-fit results.

The output produced from the software is as follows:

Best-fit values

| | |
|---|---|
| a | 63.87 |
| c | 0.003935 |
| b | −2.034 |

Std. Error

| | |
|---|---|
| a | 3.610 |
| c | 0.0003221 |
| b | 0.2989 |

95% Confidence Intervals

| | |
|---|---|
| a | 53.85 to 73.89 |
| c | 0.003040 to 0.004829 |
| b | −2.863 to −1.204 |

Goodness of Fit

| | |
|---|---|
| Degrees of Freedom | 4 |
| R² | 0.9920 |
| Absolute Sum of Squares | 21.25 |
| Sy.x | 2.305 |

Data

| | |
|---|---|
| Number of X values | 7 |
| Number of Y replicates | 1 |
| Total number of values | 7 |
| Number of missing values | 0 |

The “best fit values” are the “a, b, and c” values used in all the equations described herein. Once the “a, b, and c” values are determined, proceed with the following equations.

In order to mathematically understand the point where the perceived odor intensity reaches its maximum for each given perfume material, the derivative of the Intensity is calculated:
d(I)/d(x)=d(a/(1+(x/c)ˆb))/d(x) (II)

Therefore,
d(I)/d(x)=(ab(x/c)ˆb)/(x((x/c)ˆb+1)ˆ2 (III)

By solving this equation the following differential is obtained:
d(intensity)/d(x)=(ab(x/c)ˆb)/(x((x/c)ˆb+1)ˆ2. (IV)

By definition, the curve that describes that Intensity and its differential curve will meet at the point where a given material delivers the maximum intensity with the minimum mass. Therefore, the next step is to calculate the intersection point by solving the following equation:
(ab(xi/c)ˆb)/(x((x/c)ˆb+1)ˆ2=a/(1+(x/c)ˆb) (V)

Therefore,
(ab(xi/c)ˆb)/(xi((xi/c)ˆb+1)ˆ2−a/(1+(xi/c)ˆb)=0 (VI)

Once the value of “xi” is known, calculate the value of the Intensity at the intersection (Ii) by using the following equation: Ii=a/(I+(xi/c)ˆb) (VII)

Finally, calculate the HRSF using the equation from above: HRSF=Ii/xi (VIII)

Example calculations of human recognition slope factor are as follows (for purposes of this example they are in a low hydrophobic media (DPG)):

| Material | xi | Ii | HRSF |
|---|---|---|---|
| Ionone Beta | 0.0031 | 47.90 | 15,451 |
| Ambroxan | 0.0089 | 68.31 | 7,675 |
| Melonal | 0.0101 | 63.67 | 6,304 |
| Linalool | 0.0118 | 56.95 | 4,826 |
| Prenyl Acetate | 0.0205 | 72.55 | 3,539 |
| Geranyl nitrile | 0.0196 | 59.16 | 3,018 |
| Ethyl-2-methyl- | 0.0317 | 92.73 | 2,925 |
| butyrate | | | |
| Ligustral | 0.0249 | 70.96 | 2,850 |
| Phenyl Acetaldehyde | 0.0312 | 85.68 | 2,746 |
| Indol | 0.0312 | 77.51 | 2,484 |
| Fructone | 0.0278 | 62.36 | 2,243 |
| Alpha Damascone | 0.0309 | 62.86 | 2,034 |
| Cis 3 Hexenyl Acetate | 0.0522 | 88.84 | 1,702 |
| Lauric Ald | 0.0478 | 67.33 | 1,409 |
| Tricyclo decenyl | 0.0339 | 46.74 | 1,379 |
| acetate | | | |
| Para Cresyl Methyl | 0.0651 | 86.07 | 1,322 |
| Ether | | | |
| 7-acetyl, | 0.0473 | 58.59 | 1,239 |
| 1,2,3,4,5,6,7,8- | | | |
| octahydro-1,1,6,7- | | | |
| tetramethyl | | | |
| naphthalene | | | |
| 3-buten-2-one, 3- | 0.071 | 85.50 | 1,204 |
| methyl-4-(2,6,6- | | | |
| trimethyl-2- | | | |
| cyclohexen-1-yl) | | | |
| Cyclogalbanate | 0.0771 | 90.32 | 1,171 |
| Ionone Apha | 0.0795 | 91.99 | 1,157 |
| Decyl Aldehyde | 0.0495 | 54.35 | 1,098 |
| Calone 1951 | 0.0698 | 71.88 | 1,030 |
| Geraniol | 0.072 | 73.85 | 1,026 |
| Verdox | 0.052 | 51.91 | 998 |
| Allyl Caproate | 0.0969 | 87.81 | 906 |
| Anisic Aldehyde | 0.0946 | 83.84 | 886 |
| Florhydral | 0.06375 | 55.07 | 864 |
| Iso Eugenol | 0.0853 | 72.11 | 845 |
| Tetra Hydro linalool | 0.0641 | 53.69 | 838 |
| Methyl Cedrylone | 0.1041 | 84.44 | 811 |
| D-Limonene | 0.0847 | 67.67 | 799 |
| Methyl Phenyl | 0.067 | 50.63 | 756 |
| Carbinyl Acetate | | | |
| Methyl | 0.0737 | 54.97 | 746 |
| dihydrojasmonate | | | |
| Frutene | 0.1089 | 80.35 | 738 |
| Benzaldehyde | 0.1091 | 77.81 | 713 |
| Citral | 0.0911 | 64.84 | 712 |
| Polysantol | 0.106 | 73.16 | 690 |
| Eugenol | 0.0795 | 53.63 | 675 |
| Citronellol | 0.0702 | 45.56 | 649 |

Preferred high odor value materials useful in the perfume booster accords of the present invention include, but are not limited to: 4-(2,6,6-Trimethyl-1-cyclohexenyl)-3-butenone-2; (3aR-(3aalpha,5abeta,9aalpha,9bbeta))-dodecahydro-3a,6,6,9a-tetramethyl naphtha(2,1-b)furan; 2,6-Dimethyl-5-heptenal; 3,7-Dimethyl-1,6-octadien-3-ol; 3-Methyl-2-buten-1-yl acetate; 3,7-Dimethyl-2,6-octadienenitrile; Ethyl-2-methyl-butyrate; 2,4-Dimethylcyclohexene-3-carbaldehyde; Phenyl Acetaldehyde, Indol, ethyl methyl dioxolane acetate; 4-(2,6,6-Trimethyl-1,3-cyclohexadienyl)-3-buten-4-one; Cis 3 Hexenyl Acetate; Lauric Ald, Tricyclo decenyl acetate, Para cresyl methyl ether, 7-acetyl, 1,2,3,4,5,6,7,8-octahydro-1,1,6,7-tetramethyl naphthalene; 3-buten-2-one; 3-methyl-4-(2,6,6-trimethyl-2-cyclohexen-1-yl); Acetic acid (Cyclohexyloxy), 2-propenyl ester; 3-buten-2-one, 4-(2,6,6-trimethyl-2-cyclohexen-1-yl), (E); Decyl Aldehyde, Methyl-3,4-dioxy(cylcoacetonyl) benzene; 2,6-Dimethyl-2,6-octadien-8-ol; ortho tertiary butyl cyclohexanyl acetate; Hexanoic acid,2-propenyl ester; 4-Methoxybenzaldehyde; 3-(3-Isopropylphenyl)butanal; Iso 2-Methoxy-4-(2-propenyl)phenol, Tetra Hydro 3,7-Dimethyl-1,6-octadien-3-ol; Methyl cedrylone; 1-methyl-4-isopropenyl-1-cyclohexene; Methyl phenyl carbonyl acetate; Methyl dihydro jasmonate; Hexahydro-4,7methano-1H-inden-5(or 6)-yl propionate; Benzaldehyde, 3,7-Dimethyl-2,6-octadienal; 3,3-Dimethyl-5-(2,2,3-trimethyl-3-cycloenten-1-yl)-4-penten-2-ol; 2-Methoxy-4-(2-propenyl)phenol; 3,7-dimethyl-6-octen-1-ol; undecalactone; Allyl heptanoate; 1,3-Oxathiane, 2-methyl-4-propyl-, cis-; paradiff; (all-E)-alpha-sinensal 2,6,10-trimethyl-2(E),6(E),9(E), 11-dodecatetraenal; mandarin aldehyde, p-1-menthen-8thiol; Beta gamma hexenol; 4-Methyl-3-decen-5-ol; Ethyl caproate, Ethyl-2-4-decadienoate, 4-Penten-1-one, 1-(5,5-Dimethyl-1-cyclohexen-1-yl)-; 1H-Indene-a-propanal, 2,3-dihydro-1,1-dimethyl-(9CI); Methyl nonyl acetaldehyde; Orange juice Carbonyls; 4 dodecenal; 3-cyclohexene-1-carboxaldehyde, 2,4-dimethyl; 2,6,-nonenol; 2,6-nonadeinal; 2,6-nonadienol; Amyl acetate; 3-P-cumenyl-propionaldehyde 4-(1-methylethyl)-benzenepropanal; 1-(2,6,6-Trimethyl-1,3-cyclohexandienyl)-2-buten-1-one; 6-(Z,3-pentenyl)-tetrahydro-(2H)-pyranone-2; and 3-Methyl-(cis-2-penten-1-yl)-2-cyclopenten-1-one.

One example of a preferred perfume booster accord is comprised of the following high odor value materials: 2,6 nonenol; 2,6, nonadienal; 2,6-nonadienol; (3aR-(3aalpha,5abeta,9aalpha,9bbeta))-dodecahydro-3a,6,6,9a-tetramethyl naphtha(2,1-b)furan; Amyl Acetate; Beta Gamma Hexenol; Cis 3 Hexenyl Acetate; 3-P-cumenyl-propionaldehyde 4-(1-methylethyl)-benzenepropanal; 1-(2,6,6-Trimethyl-1,3-cyclohexandienyl)-2-buten-1-one; Ethyl-2-methyl butyrate; 3-(3-Isopropylphenyl)butanal; 4-Penten-1-one, 1-(5,5-Dimethyl-1-cyclohexen-1-yl)-; 1H-Indene-a-propanal, 2,3-dihydro-1,1-dimethyl-(9CI); 4-(2,6,6-Trimethyl-1-cyclohexenyl)-3-butenone-2; 6-(Z,3-pentenyl)-tetrahydro-(2H)-pyranone-2; 2,6-Dimethyl-5-heptenal; 6,6-Dimethylbicyclo {3.1.1}Hept-2-ene-2-proponal; 3-cyclohexene-1-carboxaldehyde, 2,4-dimethyl; Undecalactone; 4-Methyl-3-decen-5-ol; ortho tertiary butyl cyclohexanyl acetate; 3-Methyl-(cis-2-penten-1-yl)-2-cyclopenten-1-one; and 4-Pentene-2-ol, 3,3-dimethyl-5-(2,2,3-trimethyl-3-cyclopenten-1-yl)-.

Another example of a preferred perfume booster accord is comprised of the following high odor value materials: Benzaldehyde; Undeclactone; 4-(2,6,6-Trimethyl-1-cyclohexenyl)-3-butenone-2; Allyl Heptanoate; 1,3-Oxathiane, 2-methyl-4-propyl-, cis-; Paradiff, (all-E)-alpha-sinensal 2,6,10-trimethyl-2(E),6(E),9(E), 11-dodecatetraenal; mandarin aldehyde; 4-dodecenal; p-1-menthen-8 thiol; Beta Gamma Hexenol, Cis 3 Hexenyl Acetate, Orange Juice Carbonyls; ortho tertiary butyl cyclohexanyl acetate; Decyl Aldehyde; 4-Methyl-3-decen-5-ol; and 4-Penten-1-one, 1-(5,5-Dimethyl-1-cyclohexen-1-yl)-.

Still another example of a preferred perfume booster accord is comprise of the following high odor value materials: Hexanoic acid,2-propenyl ester; 4-Methoxybenzaldehyde; Allyl Heptanoate; Benzaldehyde; Beta Gamma Hexenol; 1,3-Oxathiane, 2-methyl-4-propyl-, cis-; Cis 3 Hexenyl Acetate; Decyl Aldehyde; Ethyl 2′4-decadienoate; Ethyl Caproate; Ethyl-2-methyl Butyrate; 4-Penten-1-one, 1-(5,5-Dimethyl-1-cyclohexen-1-yl)-; p-1-menthen-8 thiol; (all-E)-alpha-sinensal 2,6,10-trimethyl-2(E),6(E),9(E), 11-dodecatetraenal; 1H-Indene-a-propanal, 2,3-dihydro-1,1-dimethyl-(9CI); 4-(2,6,6-Trimethyl-1-cyclohexenyl)-3-butenone-2; 3 dodecenal; Methyl Nonyl Acetaldehyde; Orange Juice Carbonyls; Paradiff; undecalactone; 4 dodecenal; 3-cyclohexene-1-carboxaldehyde, 2,4-dimethyl; 4-Methyl-3-decen-5-ol; and ortho tertiary butyl cyclohexanyl acetate.

In some embodiments, the perfume booster accord is specifically not encapsulated. In other embodiments, the perfume booster accord is encapsulated. A wide variety of capsules exist which will allow for delivery of perfume effect at various times. Examples of such capsules with different encapsulated materials are capsules provided by microencapsulation. One method comprises a capsule core which is coated completely with a material which may be polymeric. U.S. Pat. No. 4,145,184 and U.S. Pat. No. 4,234,627.

B. Parent Perfume Solution

Compositions of the present invention may optionally comprise a parent perfume solution (may or may not be encapsulated) in addition to the perfume booster accord. The optional parent perfume solution will generally include a fragrance material. As used herein, the term “fragrance” is used to indicate any odiferous material. Any fragrance material suitable for use in cosmetic compositions may be used herein. Exemplary fragrance materials are liquid at room temperature. Generally, the fragrance material will be present at a level of from about 0.01% to about 15%, by weight of the total composition. When present, the fragrance material is preferably present at a level of from about 0.05% to about 10%, and more preferably present at a level of from about 0.1% to about 5%, by weight of the composition.

A wide variety of chemicals are known for fragrance uses, including materials such as aldehydes, ketones and esters. More commonly, naturally occurring plant and animal oils and exudates comprising complex mixtures of various chemical components are known for use as fragrances. The fragrances herein can be relatively simple in their compositions, comprising a single chemical, or can comprise highly sophisticated complex mixtures of natural and synthetic chemical components, all chosen to provide any desired odour.

Preferably the fragrance materials of the present invention will have boiling points (BP) of about 500° C. or lower, more preferably about 400° C. or lower, even more preferably about 350° C. or lower. The BP of many fragrance materials are given in Perfume and Flavor Chemicals (Aroma Chemicals), Steffen Arctander (1969). The ClogP value of the fragrance materials useful herein is preferably greater than about 0.1, more preferably greater than about 0.5, even more preferably greater than about 1.0, even more preferably still greater than about 1.2.

Suitable fragrance materials can be found in the following U.S. Pat. Nos. 4,145,184; 4,209,417; 4,515,705; and 4,152,272. Examples of fragrances useful herein include, but are not limited to: animal fragrances such as musk oil, civet, castoreum, ambergris; plant fragrances such as nutmeg extract, cardomon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract, carrot seed extract, jasmine extract, olibanum extract, rose extract, and mixtures thereof.

Other examples of suitable fragrance materials include, but are not limited to: chemical substances such as acetophenone, adoxal, aldehyde C-12, aldehyde C-14, aldehyde C-18, allyl caprylate, allyl heptanoate, ambroxan, amyl acetate, dimethylindane derivatives, α-amylcinnamic aldehyde, anethole, anisaldehyde, benzaldehyde, benzyl acetate, benzyl alcohol and ester derivatives, benzyl propionate, benzyl salicylate, beta gamma hexanol, bomeol, butyl acetate, camphor, carbitol, carvone, cetalox, cinnamaldehyde, cinnamyl acetate, cinnamyl alcohol, cis-3-hexanol and ester derivatives, cis-3-hexenyl methyl carbonate, cis jasmone, citral, citronnellol and ester derivatives, cumin aldehyde, cyclamen aldehyde, cyclo galbanate, damascones, decalactone, decanol, decyl aldehyde, estragole, delta muscenone, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl 2 Methyl Butyrate, ethyl maltol, ethyl propionate, ethyl caprylate, ethyl cinnamate, ethyl hexanoate, ethyl valerate, ethyl vanillin, eugenol, exaltolide, fenchone, floralozone, fruity esters such as ethyl 2-methyl butyrate, galaxolide, gamma decalactone, geraniol and ester derivatives, hedione, helional, 2-heptonone, hexenol, hexyl acetate, hexyl salicylate, α-hexylcinnamic aldehyde, p-hydroxy phenyl butanone, hydroxycitrolnellal, indole, isoamyl acetate, isoeugenol acetate, ionones, isoeugenol, isoamyl iso-valerate, iso E super, limonene, linalool, linalool acteate, lilial, linalyl acetate, lyral, majantol, mayol, melonal, menthol, p-methylacetophenone, methyl anthranilate, methyl cedrylone, methyl dihydrojasmonate, methyl eugenol, methyl ionone, methyl-β-naphthyl ketone, methylphenylcarbinyl acetate, mugetanol, γ-nonalactone, 2-6 nonedienal, octanal, para hydroxy phenyl butanone, polysantol, phenoxynol, phenyl ethyl acetate, phenyl-acetaldehyde dimethyl acetate, phenoxyethyl isobutyrate, phenyl ethyl alcohol, pinenes, sandalore, sanjinol, santalol, stemone, thymol, terpenes, tonalide, triplal, triethyl citrate, 3,3,5-trimethylcyclohexanol, γ-undecalactone, undecavertol, undecenal, undecylenic aldehyde, vanillin, veloutone, verdox and mixtures thereof.

C. Compositions

The personal care compositions of the present invention may be selected from the group consisting of shampoo, conditioner, antiperspirant, deodorant, hair styling products, cleansers, soaps, bar soap, bodywash, cosmetics, foundations, lotions, creams, ointments, and hydro-alcoholic solutions.

In one embodiment, the personal care composition may be a multi-phase composition and comprises at least two physically distinct phases, preferably visually distinct phases. In a particular embodiment, the visually distinct phases are of a different color, or one phase may be visibly clear. For instance, one or more phases can comprise a dye, pigment, pearlescent agent, lake, coloring, or mixtures thereof. Colorants useful in the present invention can be, for example, selected from the group consisting of Red 30 Low Iron, FD&C Red 40 AL Lake, D&C Red Lake Blend of Lake 27 & Lake 30, FD&C Yellow 5 A1 Lake, FD&C Yellow 6 A1 Lake, FD&C Yellow 5 Lake, FD&C Blue #1 AL Lake, Kowet Titanium Dioxide, D&C Red 30 Talc Lake, D&C Red 6 Barium Lake, D&C Red 7 Calcium Lake, D&C Red 34 Calcium Lake, D&C Red 30 AL lake, D&C Red 27 AL lake, D&C Yellow 10 AL lake, D&C Red 21 AL Lake, Yellow Iron Oxide, D&C Red 30 Lake, Octocir Yellow 6 AL Lake, Octocir Yellow 5 AL Lake, D&C Red 28 Lake, D&C Orange 5 Zirc Al Lake, Cos Red Oxide BC, Cos Iron Oxide Red BC, Cos Iron oxide Black BC, Cos Iron Oxide Yellow, Cos Iron Oxide Brown, Cos Iron Oxide Yellow BC, Euroxide Red Unsteril, Euroxide Black Unsteril, Euroxide Yellow Steril, Euroxide Black Steril, Euroxide Red, Euroxide Black, Hydrophobic Euroxide Black, Hydrophobic Euroxide Yellow, Hydrophobic Euroxide Red, D&C Yellow 6 Lake, D&C Yellow 5 Zr Lake, and mixtures thereof.

The personal care composition of the present invention may comprise an aqueous carrier, preferably water. The personal care composition of the present invention may also comprise conditioning agents such as hydrolysed collagen with tradename Peptein 2000 available from Hormel, water soluble and water insoluble vitamins such as vitamin A, D, B1, B2, B6, B12, C, biotin, vitamin E with tradename Emix-d available from Eisai, panthenol available from Roche, pantothenic acid, panthenyl ethyl ether available from Roche, and their derivatives; hydrolysed keratin, proteins, plant extracts, and nutrients; emollients such as PPG-3 myristyl ether with tradename Varonic APM available from Goldschmidt, Trimethyl pentanol hydroxyethyl ether, PPG-11 stearyl ether with tradename Varonic APS available from Goldschmidt, Stearyl heptanoate with tradename Tegosoft SH available from Goldschmidt, Lactil (mixture of Sodium lactate, Sodium PCA, Glycine, Fructose, Urea, Niacinamide, Glucosamine, Inositol, Sodium Benzoate, and Lactic acid) available from Goldschmidt, Sodium lactate, Sodium PCA, Glycine, Fructose, Urea, Niacinamide, Glucosamine, Inositol, Sodium Benzoate, Lactic acid, Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, and silicone grafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes, oxidative dyes and interference pigments; hair oxidizing (bleaching) agents, such as hydrogen peroxide, perborate and persulfate salts, carbonate; hair reducing agents such as the thioglycolates; perfumes; and sequestering agents, such as disodium ethylenediamine tetra-acetate; ultraviolet and infrared screening and absorbing agents such as octyl salicylate; antimicrobial agents; suspending agents; viscosity modifiers; nonvolatile solvents or diluents (water soluble and insoluble), pearlescent aids, foam boosters, additional surfactants or nonionic cosurfactants, pediculocides, chelants, skin active agents, sunscreens, UV absorbers, and, water soluble and insoluble amino acids such as asparagine, alanin, indole, glutamic acid, tyrosine, tryptamine, and their salts; and antidandruff agents such as zinc pyrithione, pyridinethione salts, azoles, climbazole, octopirox, salicylic acid, selenium sulfide, particulate sulfur, and mixtures thereof.

### Method of Use

The personal care compositions of the present invention are used in conventional ways on hair and skin. Such method of use depends upon the type of composition employed but generally involves application of an effective amount of the product to the hair or skin, which may then be rinsed from the hair or skin (as in the case of hair rinses) or allowed to remain on the hair or skin (as in the case of gels, lotions, and creams). “Effective amount” means an amount sufficient enough to provide a dry combing benefit. In general, from about 1 g to about 50 g is applied to the hair, skin, or the scalp. The composition is distributed throughout the hair or skin, typically by rubbing or massaging the hair, scalp, or skin. Preferably, the composition is applied to wet or damp hair prior to drying of the hair. After such compositions are applied to the hair, the hair is dried and styled in accordance with the preference of the user. In the alternative, the composition is applied to dry hair, and the hair is then combed or styled in accordance with the preference of the user. The personal care compositions are useful in delivering conditioning benefits to hair or skin, and/or delivering hair styling benefits to hair or skin, and/or delivering hair coloring benefits to hair or skin by topically applying an effective amount of the composition onto hair or skin and removing said composition from said hair or skin by rinsing with water.

### Method of Making

The personal care compositions of the present invention may be prepared by any known or otherwise effective technique, suitable for making and formulating the desired product form.

### NON-LIMITING EXAMPLES

The compositions illustrated in the following Examples show personal care compositions comprising the perfume booster accord of the present invention. The following Examples exemplify specific embodiments of the compositions of the present invention, but are not intended to be limiting thereof. Other modifications can be undertaken by the skilled artisan without departing from the spirit and scope of this invention.

The compositions illustrated in the following Examples are prepared by conventional formulation and mixing methods, an example of which is described above. All exemplified amounts are listed as weight percents and exclude minor materials such as diluents, preservatives, color solutions, imagery ingredients, botanicals, and so forth, unless otherwise specified.

### Examples 1-10

### Multi-Phase Shampoo Composition

| | 1 | 2 | 3 | 4 | 5 | 6 | 7 | 8 | 9 | 10 |
|---|---|---|---|---|---|---|---|---|---|---|
| Phase A Composition | | | | | | | | | | |
| Ammonium Laureth-3 Sulfate | | | | 12 | | | 6 | 6 | 8 | |
| Ammonium Lauryl Sulfate | | | | 2 | | | 10 | 10 | 10 | |
| Sodium Laureth-3 Sulfate | 6 | 8 | 6 | | 6 | 10 | | | | 10 |
| Sodium Lauryl Sulfate | 10 | 8 | 10 | | 10 | 6 | | | | 7 |
| Cocamidopropyl Betaine FB | | | | 2 | | | | | | |
| Sodium Lauraoamphoacetate | | | | 1 | | | | | | |
| Cocamide MEA | 0.8 | 0.8 | 1.5 | 0.8 | 0.8 | 0.8 | 1.35 | 1.35 | 0.8 | 0.8 |
| Ethylene Glycol Distearate | | | | 1.5 | | 2 | | | 3 | |
| Cetyl Alcohol | | | 0.9 | | | | | | | 0.45 |
| Polyquat 10 (1) | 0.5 | | 0.4 | | 0.5 | | | 0.5 | 0.5 | 1 |
| Guar Hydroxypropyl trimonium | | 0.4 | | 0.05 | | | 0.3 | | | |
| Chloride (2) | | | | | | | | | | |
| Polymethacrylamidopropyl | | | | | | 0.4 | | | 0.2 | |
| trimonium Chloride (3) | | | | | | | | | | |
| Carbopol Aqua SF-1 (4) | 4 | 5 | 4.5 | 4 | 4 | 5.75 | 4 | 3.5 | 5.5 | 4 |
| PEG 14M (5) | | | 0.05 | 0.15 | | 0.05 | 0.1 | 0.05 | 0.1 | 0.1 |
| Dimethicone (6) | | | | 2 | | 1 | | | 1 | 2 |
| Perfume Solution | 0.5 | 0.8 | 0.1 | 0.6 | 1 | 0.3 | 0.8 | 0.8 | 0.2 | 0.1 |
| Perfume Booster Accord | 0.25 | 0.25 | 0.5 | 0.25 | 0.05 | 0.3 | 0.25 | 0.1 | 0.25 | 0.4 |
| Methylchloroisothiazolinone/ | 0.0005 | 0.0005 | 0.0005 | 0.0005 | 0.0005 | 0.0005 | 0.0005 | 0.0005 | 0.0005 | 0.0005 |
| Methylisothiazolinone | | | | | | | | | | |
| Disodium EDTA | 0.1274 | 0.1274 | 0.1274 | 0.1274 | 0.1274 | 0.1274 | 0.1274 | 0.1274 | 0.1274 | 0.1274 |
| Sodium Benzoate | 0.25 | 0.25 | 0.25 | 0.25 | 0.25 | 0.25 | 0.25 | 0.25 | 0.25 | 0.25 |
| Citric Acid | 0.6 | 0.9 | 0.8 | 0.8 | 0.8 | 0.8 | 0.4 | 0.4 | 0.4 | 0.8 |
| Sodium Hydroxide | 0.35 | 0.5 | 0.4 | 0.35 | 0.35 | 0.5 | 0.35 | 0.5 | 0.35 | 0.35 |
| Ammonium Xylene Sulfonate | | | | | | | | 1 | | |
| Sodium Xylene Sulfonate | | 1.5 | 0.5 | | | 0.75 | | | | |
| Water and Minors (QS to 100%) | | | | | | | | | | |
| Phase B Composition | | | | | | | | | | |
| Ammonium Laureth-3 Sulfate | | | | 12 | | | 6 | 6 | 8 | |
| Ammonium Lauryl Sulfate | | | | 2 | | | 10 | 10 | 10 | |
| Sodium Laureth-3 Sulfate | 6 | 8 | 6 | | 10 | 6 | | | | 10 |
| Sodium Lauryl Sulfate | 10 | 8 | 10 | | 6 | 10 | | | | 7 |
| Cocamidopropyl Betaine FB | | | | 2 | | | | | | |
| Sodium Lauraoamphoacetate | | | | 1 | | | | | | |
| Cocamide MEA | 0.8 | 0.8 | 1.5 | 0.8 | 0.8 | 0.8 | 1.35 | 1.35 | 0.8 | 0.8 |
| Ethylene Glycol Distearate | | | 1.5 | | | 2 | | | | |
| Cetyl Alcohol | | | 0.9 | | | | | | | 0.45 |
| Polyquat 10 (1) | 0.25 | | 0.1 | | 0.25 | | | 0.5 | 0.05 | 0.1 |
| Guar Hydroxypropyl trimonium | | 0.25 | | 0.05 | | | 0.2 | | | |
| Chloride (2) | | | | | | | | | | |
| Polymethacrylamidopropyl | | | | | | 0.4 | | | 0.2 | |
| trimonium Chloride (3) | | | | | | | | | | |
| Carbopol Aqua SF-1 (4) | 4 | 5 | 4.5 | 4 | 4 | 5.75 | 4 | 3.5 | 5.5 | 4 |
| PEG 14M (5) | | | 0.05 | 0.15 | | 0.05 | 0.1 | 0.05 | 0.1 | 0.1 |
| Dimethicone (6) | | | 2 | | 1.5 | 1 | | | 1 | 2 |
| Perfume Solution | 0.5 | 0.8 | 0.1 | 0.6 | 1 | 0.3 | 0.8 | 0.8 | 0.2 | 0.1 |
| Perfume Booster Accord | 0.25 | 0.25 | 0.5 | 0.25 | 0.05 | 0.3 | 0.25 | 0.1 | 0.25 | 0.4 |
| Methylchloroisothiazolinone/ | 0.0005 | 0.0005 | 0.0005 | 0.0005 | 0.0005 | 0.0005 | 0.0005 | 0.0005 | 0.0005 | 0.0005 |
| Methylisothiazolinone | | | | | | | | | | |
| Disodium EDTA | 0.1274 | 0.1274 | 0.1274 | 0.1274 | 0.1274 | 0.1274 | 0.1274 | 0.1274 | 0.1274 | 0.1274 |
| Sodium Benzoate | 0.25 | 0.25 | 0.25 | 0.25 | 0.25 | 0.25 | 0.25 | 0.25 | 0.25 | 0.25 |
| Citric Acid | 0.6 | 0.9 | 0.8 | 0.8 | 0.8 | 0.8 | 0.4 | 0.4 | 0.4 | 0.8 |
| Sodium Hydroxide | 0.35 | 0.5 | 0.4 | 0.35 | 0.35 | 0.5 | 0.35 | 0.5 | 0.35 | 0.35 |
| Ammonium Xylene Sulfonate | | | | | | | | 1 | | |
| Sodium Xylene Sulfonate | | 1.5 | 0.5 | | | 0.75 | | | | |
| D&C Red #30 Talc Lake | 0.02 | 0.05 | 0.05 | 0.05 | 0.05 | 0.05 | 0.05 | 0.05 | 0.02 | 0.05 |
| Water and Minors (QS to 100%) | | | | | | | | | | |
| Ratio of Phase A to Phase B | 70:30 | 67:33 | 50:50 | 10:90 | 33:67 | 90:10 | 67:33 | 60:40 | 90:10 | 60:40 |

(1) Polymer LR30M available from Amerchol/Dow Chemical
(2) Jaguar C17 available from Rhodia
(3) Polycare 133 available from Rhodia
(4) Carbopol Aqua SF-1 available from Noveon
(5) PEG14M, WSR N-3000 available from Union Carbide/Dow Chemical
(6) Viscasil 330M available from General Electric Silicones

### Examples 11-15

### Shampoo Composition

| | 11 | 12 | 13 | 14 | 15 |
|---|---|---|---|---|---|
| Ammonium Laureth-3 Sulfate | | | 10 | 12 | 8 |
| Ammonium Lauryl Sulfate | | | 6 | 2 | 8 |
| Sodium Laureth-3 Sulfate | 6 | 8 | | | |
| Sodium Lauryl Sulfate | 10 | 6 | | | |
| Cocamidopropyl Betaine FB | | 2 | | 2 | |
| Cocamide MEA | 0.8 | 0.5 | | | |
| Ethylene Glycol Distearate | | | 1.5 | 2 | 1.5 |
| Cetyl Alcohol | | | 0.9 | 0.9 | 0.9 |
| Polyquat 10(1) | 0.25 | | 0.5 | 0.25 | |
| Carbopol Aqua SF-1(2) | 2 | | | | |
| Dimethicone(3) | | | 1.5 | 1 | 1.5 |
| Perfume Solution | 0.7 | 0.6 | 0.4 | 0.35 | 0.1 |
| Perfume Booster Accord | 0.05 | 0.3 | 0.3 | 0.35 | 0.5 |
| Methylchloroisothiazolinone/ | 0.0005 | 0.0005 | 0.0005 | 0.0005 | 0.0005 |
| Methylisothiazolinone | | | | | |
| Disodium EDTA | 0.1274 | 0.1274 | 0.1274 | 0.1274 | 0.1274 |
| Sodium Benzoate | 0.25 | 0.25 | 0.25 | 0.25 | 0.25 |
| Citric Acid | 0.6 | 0.4 | 0.4 | 0.4 | 0.4 |
| Sodium Hydroxide | 0.35 | | | | |
| Sodium Chloride | | 0.75 | 0.6 | 0.6 | 0.6 |
| Ammonium Xylene Sulfonate Water and | | | 0.2 | | |
| Minors (QS to | | | | | |
| 100%) | | | | | |

(1) Polymer LR30M available from Amerchol/Dow Chemical
(2) Carbopol Aqua SF-1 available from Noveon
(3) Viscasil 330M available from General Electric Silicones

### Examples 16-31

### Multi-Phase Conditioner Composition

### Examples 16-18

| | Example | Example | |
|---|---|---|---|
| | 16 | 17 | Example 18 |
| Conditioning Phase | | | |
| Composition | | | |
| Stearamidopropyldimethylamine | 2.0 | 2.0 | 3.0 |
| (1) | | | |
| L-Glutamic acid (2) | 0.64 | 0.64 | 0.96 |
| Cetyl alcohol (3) | 2.5 | 3.75 | 3.75 |
| Stearyl alcohol (4) | 4.5 | 6.75 | 6.75 |
| Dimethicone blend (5) | — | — | 6.3 |
| Dimethicone/Cyclomethicone | 4.2 | 4.2 | |
| blend (6) | | | |
| Benzyl alcohol (7) | 0.4 | 0.4 | 0.4 |
| EDTA (8) | 0.1 | 0.1 | 0.1 |
| Kathon CG (9) | 0.03 | 0.03 | 0.03 |
| Panthenyl Ethyl Ether (10) | 0.05 | 0.06 | 0.06 |
| Panthenol (11) | 0.09 | 0.09 | 0.05 |
| Perfume Booster Accord | 0.25 | 0.20 | 0.25 |
| Deionized Water | qs | qs | qs |
| Benefit Phase Composition | | | |
| Behetrimonium Chloride (13) | 3.38 | 2.25 | 4.05 |
| Cetyl alcohol | 2.32 | 1.86 | 2.5 |
| Stearyl alcohol | 4.18 | 4.64 | 4.5 |
| Polysorbate - 60 (14) | 0.2 | 0.2 | 0.1 |
| Amino-silicone (15) | 2.0 | 0.5 | |
| Amino-, polyol-silicone(16) | | | 2.0 |
| Benzyl alcohol | 0.4 | 0.4 | 0.4 |
| EDTA | 0.13 | 0.13 | 0.13 |
| Kathon CG | 0.033 | 0.033 | 0.033 |
| Panthenyl Ethyl Ether | 0.05 | 0.05 | 0.05 |
| Panthenol | 0.05 | 0.05 | 0.05 |
| Sodium hydroxide | 0.014 | 0.014 | 0.014 |
| Isopropyl alcohol | 0.9 | — | 0.9 |
| Pigment (17) | 0.08 | 0.08 | 0.08 |
| Perfume Booster Accord | 0.5 | 0.5 | 0.5 |
| Deionized Water | qs | qs | qs |
| Ratio Conditioning Phase/ | 50/50 | 60/40 | 50/50 |
| Benefit Phase | | | |

(1) supplied by Inolex under trade name Lexamine S-13
(2) supplied by Ajinomoto
(3) supplied by Procter & Gamble
(4) supplied by Procter & Gamble
(5) supplied by GE Silicones as a blend of dimethicone having a viscosity of 18,000,000 mPs · s and dimethicone having a viscosity if 200 mPa · s
(6) supplied by GE Silicone as a blend of dimethicone having a viscosity if 18,000,000 mPa · s and cyclopentasiloxane
(7) supplied by Haarman & Reimer
(8) supplied by BASF
(9) supplied by Rohm & Haas
(10) supplied by Roche
(11) supplied by Roche
(13) supplied by Clariant
(14) supplied by ICI as Tween 60
(15) supplied by Wacker as ADM1100
(16) supplied by Dow Corning Corporation as reference number 17828-137
(17) supplied by Rona

### Examples 19-21

| | Example | Example | |
|---|---|---|---|
| | 19 | 20 | Example 21 |
| Conditioner | | | |
| Phase Composition | | | |
| Behetrimonium Chloride (13) | 3.38 | 2.25 | 4.05 |
| Cetyl alcohol | 2.32 | 1.86 | 2.5 |
| Stearyl alcohol | 4.18 | 4.64 | 4.5 |
| Polysorbate - 60 (14) | 0.2 | 0.2 | 0.1 |
| Amino-silicone (15) | 2.0 | 0.5 | |
| Amino-, polyol-silicone (16) | — | — | 2.0 |
| Benzyl alcohol | 0.4 | 0.4 | 0.4 |
| EDTA | 0.13 | 0.13 | 0.13 |
| Kathon CG | 0.033 | 0.033 | 0.033 |
| Panthenyl Ethyl Ether | 0.05 | 0.05 | 0.05 |
| Panthenol | 0.05 | 0.05 | 0.05 |
| Sodium hydroxide | 0.014 | 0.014 | 0.014 |
| Isopropyl alcohol | 0.9 | — | 0.9 |
| Pigment (17) | 0.08 | 0.08 | 0.08 |
| Perfume Booster Accord | 0.5 | 0.5 | 0.5 |
| Deionized Water | qs | qs | Qs |
| Benefit Phase Composition | | | |
| Pemulen TR-1 (18) | 0.35 | — | — |
| Ketrol (19) | — | 0.75 | 0.25 |
| Acrylate/Vinyl Isodecanoate | — | — | 0.75 |
| Crosspolymer (20) | | | |
| Laponite XLS (21) | — | 0.25 | — |
| Triethanolamine (22) | 0.23 | — | — |
| Glydant (23) | 0.35 | 0.30 | 0.35 |
| Pigment | 0.01 | 0.02 | 0.015 |
| Water | qs | qs | Qs |
| Ratio Conditioning Phase/Benefit | 20/80 | 30/70 | 20/80 |
| Phase | | | |

(18) supplied by Noveon
(19) supplied by CP Kelco
(20) supplied by 3V as Stayblen 30
(21) supplied by Southern Clay Products
(22) supplied by Dow chemicals
(23) supplied by Macintyre group

### Examples 22-24

| | Example | Example | Example |
|---|---|---|---|
| | 22 | 23 | 24 |
| Conditioning Phase Composition | | | |
| Stearamidopropyldimethylamine (1) | 2.0 | 2.0 | 3.0 |
| L-Glutamic acid (2) | 0.64 | 0.64 | 0.96 |
| Cetyl alcohol (3) | 2.5 | 3.75 | 3.75 |
| Stearyl alcohol (4) | 4.5 | 6.75 | 6.75 |
| Dimethicone blend (5) | — | — | 6.3 |
| Dimethicone/Cyclomethicone | 4.2 | 4.2 | |
| blend (6) | | | |
| Benzyl alcohol (7) | 0.4 | 0.4 | 0.4 |
| EDTA (8) | 0.1 | 0.1 | 0.1 |
| Kathon CG (9) | 0.03 | 0.03 | 0.03 |
| Panthenyl Ethyl Ether (10) | 0.05 | 0.06 | 0.06 |
| Panthenol (11) | 0.09 | 0.09 | 0.05 |
| Perfume Booster Accord | 0.25 | 0.20 | 0.25 |
| Deionized Water | qs | qs | qs |
| Benefit Phase Composition | | | |
| Dipropylene Glycol Dibenzoate(24) | 70 | — | — |
| Dimethicone (5) | — | 80 | — |
| 7-3105 Petrolatum HIP Emulsion(25) | — | — | 30 |
| Polyoxyethylene (20) | — | 1.0 | — |
| monolaurate (26) | | | |
| Plantaren 2000 (27) | 5.0 | — | — |
| Glycerin (28) | 5.0 | 0.75 | — |
| Water | qs | qs | — |
| Ratio Conditioning Phase/Benefit | 80/20 | 80/20 | 70/30 |
| Phase | | | |

(24) supplied by Finetex as Finsolv PG-22
(25) supplied by Dow Corning
(26) supplied by Uniqema as Tween 20
(27) supplied by Cognis
(28) supplied by Procter&Gamble Chemicals

### Examples 25-27

| | Example | Example | Example |
|---|---|---|---|
| | 25 | 26 | 27 |
| Conditioning Phase Composition | | | |
| Stearamidopropyldimethylamine (1) | 2.0 | — | 1.2 |
| Behenyl trimethylammonium chloride | — | 3.45 | — |
| (18) | | | |
| L-Glutamic acid (2) | 0.64 | — | 0.38 |
| Quaternium-18 (21) | — | — | 0.5 |
| Cetyl alcohol (3) | 2.5 | 1.97 | 2.00 |
| Stearyl alcohol (4) | 4.5 | 3.55 | 3.60 |
| Dimethicone blend (5) | — | 4.2 | 1.5 |
| Dimethicone/Cyclomethicone | 4.2 | — | — |
| blend (6) | | | |
| Benzyl alcohol (7) | 0.4 | 0.4 | 0.4 |
| EDTA (8) | 0.1 | 0.1 | 0.1 |
| Disodium EDTA (19) | — | 0.13 | — |
| Kathon CG(9) | 0.03 | 0.03 | 0.03 |
| Panthenyl Ethyl Ether (10) | 0.05 | 0.06 | 0.06 |
| Panthenol (11) | 0.09 | 0.09 | 0.05 |
| Perfume Booster Accord | 0.25 | 0.35 | 0.30 |
| Deionized Water | qs | qs | Qs |
| Benefit Phase Composition | | | |
| Behetrimonium Chloride (13) | 2.25 | — | 3.38 |
| Behenamidopropyldimethylamine | — | 2.30 | — |
| (20) | | | |
| Cetyl alcohol | 1.86 | 2.5 | 2.32 |
| Stearyl alcohol | 4.64 | 4.5 | 4.18 |
| Dimethicone/Cyclomethicone | — | 4.2 | 4.2 |
| blend (6) | | | |
| Aminosilicone (15) | 3.5 | — | — |
| C13-C16 Isoparaffin (16) | 1.5 | — | — |
| Benzyl alcohol | 0.4 | 0.4 | 0.4 |
| Disodium EDTA (19) | 0.13 | 0.13 | 0.13 |
| EDTA (8) | — | 0.1 | — |
| Kathon CG | 0.033 | 0.033 | 0.033 |
| Panthenyl Ethyl Ether | 0.05 | 0.05 | 0.05 |
| Panthenol | 0.05 | 0.05 | 0.05 |
| Sodium hydroxide | 0.014 | — | 0.014 |
| Isopropyl alcohol | 0.9 | — | 0.9 |
| Pigment (17) | 0.08 | 0.08 | 0.08 |
| Perfume Booster Accord | 0.5 | 0.5 | 0.5 |
| Deionized Water | qs | qs | qs |
| Ratio Conditioning Phase/Benefit | 20/80 | 30/70 | 20/80 |
| Phase | | | |

(1) supplied by Inolex under trade name Lexamine S-13
(2) supplied by Ajinomoto
(3) supplied by Procter & Gamble
(4 supplied by Procter & Gamble
(5) supplied by GE Silicones as a blend of dimethicone having a viscosity of 18,000,000 mPa · s and dimethicone having a viscosity if 200 mPa · s
(6) supplied by GE Silicone as a blend of dimethicone having a viscosity if 18,000,000 mPa · s and cyclopentasiloxane
(7) supplied by Haarman & Reimer
(8) supplied by BASF as Ethylene Diamine Tetracetic Acid
(9) supplied by Rohm & Haas
(10) supplied by Roche
(11) supplied by Roche
(13) supplied by Clariant
(15) supplied by GE Silicones as reference number Y-14900
(16) supplied by Nisseki as Isosol 400
(17) supplied by Rona
(18) supplied by Clariant as Genamin KDMP
(19) supplied by SCAL
(20) supplied by Croda as IncromineBB
(21) supplied by Goldschmidt

### Examples 28-30

| | Example | Example | |
|---|---|---|---|
| | 28 | 29 | Example 30 |
| Conditioning Phase | | | |
| Composition | | | |
| Acrylate/Aminoacrylate/ | 2.0 | — | — |
| Vinylalkoxylate/C10-30 | | | |
| Alkyl PEG-25 Methacrylate (1) | | | |
| Guar Hydroxypropyltrimonium | — | 0.70 | — |
| chloride (3) | | | |
| Hydroxypropyl guar(4) | — | 1.2 | — |
| Hydrophobically modified | — | — | 0.65 |
| cationic cellulose | | | |
| polymer (22) | | | |
| POE(10) cetyl ether(2) | 3.0 | — | — |
| Cetyl trimethylammonium | — | 1.0 | 0.8 |
| chloride (20) | | | |
| Hydrophobically modified | 4.0 | 2.0 | 3.0 |
| amidomethicone | | | |
| copolyol (5) | | | |
| Lactic Acid | 0.39 | — | — |
| Disodium EDTA (18) | 0.13 | 0.13 | 0.13 |
| Kathon CG (9) | 0.03 | 0.03 | 0.03 |
| Methyl Paraben(19) | 0.2 | 0.2 | 0.2 |
| Panthenyl Ethyl Ether(10) | — | 0.03 | 0.06 |
| Panthenol (11) | — | 0.03 | 0.05 |
| Perfume Booster Accord | 0.50 | 0.40 | 0.25 |
| Deionized Water | qs | qs | qs |
| Benefit Phase Composition | | | |
| Behetrimonium Chloride (13) | 2.25 | — | — |
| Cetyl trimethylammonium | — | 3.00 | 1.00 |
| chloride (20) | | | |
| Cetyl alcohol | 1.86 | 1.86 | 2.50 |
| Stearyl alcohol | 4.64 | 4.64 | 4.50 |
| Dimethicone blend (21) | — | 4.2 | 2.0 |
| Aminosilicon (15) | 3.50 | — | — |
| C13-C16 Isoparaffin (16) | 1.50 | — | — |
| Benzyl alcohol | 0.4 | 0.4 | 0.4 |
| Disodium EDTA (18) | 0.13 | 0.13 | 0.13 |
| Kathon CG | 0.033 | 0.033 | 0.033 |
| Panthenyl Ethyl Ether | 0.05 | 0.05 | 0.05 |
| Panthenol | 0.05 | 0.05 | 0.05 |
| Sodium hydroxide | 0.014 | 0.014 | 0.014 |
| Isopropyl alcohol | 0.9 | — | — |
| Pigment (17) | 0.08 | 0.08 | 0.08 |
| Perfume Booster Accord | 0.5 | 0.5 | 0.5 |
| Deionized Water | qs | qs | qs |
| Ratio Conditioning Phase/Benefit | 20/80 | 30/70 | 20/80 |
| Phase | | | |

(1) supplied by Noveon as reference number EX-832
(2) supplied by Nikkol as BT-10TX
(3) supplied by Rhodia as Jaguar Excel
(4) supplied by Rhodia as HP105
(5) supplied by Dow Corning as BY16-906
(6) supplied by GE Silicone as a blend of dimethicone having a viscosity if 18,000,000 mPa · s and cyclopentasiloxane
(7) supplied by Haarman & Reimer
(8) supplied by BASF
(9) supplied by Rohm & Haas
(10) supplied by Roche
(11) supplied by Roche
(13) supplied by Clariant
(14) supplied by ICI as Tween 60
(15) supplied by GE Silicones as reference number Y-14900
(16) supplied by Nisseki as Isosol 400
(17) supplied by Rona
(18) supplied by Ciba
(19) supplied by Protameen
(20) supplied by KCI
(21) supplied by GE Silicones as a blend of dimethicone having a viscosity of 18,000,000 mPa · s and dimethicone having a viscosity if 200 mPa · s
(22) supplied by Amerchol as reference number 3779-14

### Examples 31

| | Example 31 |
|---|---|
| Conditioning Phase Composition | |
| Stearamidopropyldimethylamine | 1.0 |
| Cetyl alcohol | 0.96 |
| Stearyl alcohol | 0.64 |
| Dimethicone/Cyclomethicone blend | 4.2 |
| Benzyl alcohol | 0.4 |
| Quaternium-18 (1) | 0.75 |
| PEG-2M (Polyox WAR N-10) (2) | 0.5 |
| Emulsifying Wax (Polawax NF) (3) | 0.5 |
| Hydroxyethylcellulose (HEC) (4) | 0.25 |
| Glyceryl Monostearate (GMS) (5) | 0.25 |
| Oleyl Alcohol (6) | 0.25 |
| Citric Acid (7) | 0.13 |
| EDTA | 0.1 |
| Kathon CG | 0.03 |
| Panthenyl Ethyl Ether | 0.05 |
| Panthenol | 0.09 |
| Perfume Booster Accord | 0.25 |
| Deionized Water | qs |
| Benefit Phase Composition | |
| Ketrol | 0.25 |
| Acrylate/Vinyl Isodecanoate Crosspolymer | 0.75 |
| Glydant | 0.35 |
| Pigment | 0.015 |
| Water | Qs |
| Ratio Conditioning Phase/Benefit Phase | 40/60 |

(1) supplied by Degussa as Varisoft DHT
(2) supplied by Amerchol as Polyox WSR N-10
(3) supplied by Croda
(4) supplied by Aqualon as Natrosol 250 HHR
(5) supplied by Uniqema as Arlacel 129
(6) supplied by Croda as Novol
(7) supplied by Roche Vitamins

### Examples 32-40

### Conditioner Compositions

### Examples 32-34

| | 32 | 33 | 34 |
|---|---|---|---|
| Hydroxypropyl Guar | 1.2 | 1.4 | 1.4 |
| Polyquaternium-10 | 0.7 | | |
| Polyquaternium-4 | | 0.7 | 0.7 |
| Cetrimonium Chloride | 1.0 | 1.0 | 1.0 |
| PEG-12 Methyl Ether Lauroxy PEG-5 | 2.0 | 2.0 | 2.0 |
| Amidopropyl Dimethicone | | | |
| Disodium EDTA | 0.127 | 0.127 | 0.127 |
| Methyl Paraben | 0.2 | 0.2 | 0.2 |
| Methylchloroisothiazolinone/ | 0.00495 | 0.00495 | 0.00495 |
| Methylisothiazolinone | | | |
| Perfume Booster Accord | 0.7 | 0.7 | 0.8 |
| Water and Minors (QS to 100%) | | | |

### Examples 35-40

| | 35 | 36 | 37 | 38 | 39 | 40 |
|---|---|---|---|---|---|---|
| Glutamic Acid | 0.640 | 0.640 | | | | |
| Stearamidopropyl Dimethylamine | 2.000 | | | | | |
| Behenamidopropyl Dimethylamine | | 2.300 | | | | |
| Behentrimonium chloride/Isopropyl | | | 3.381 | 3.445 | 2.250 | 2.250 |
| alcohol | | | | | | |
| Cetyl Alcohol | 2.500 | 2.500 | 2.320 | 1.972 | 1.857 | 1.857 |
| Stearyl Alcohol | 4.500 | 4.500 | 4.180 | 3.553 | 4.643 | 4.643 |
| Dimethicone, Cyclopentasiloxane | 4.200 | 4.200 | | | | |
| Dimethicone | | | 1.500 | 4.200 | | 4.200 |
| Amodimethicone | | | | | 3.500 | |
| EDTA | 0.100 | 0.100 | | | | |
| Disodium Ethylene Diamine | | | 0.127 | 0.127 | 0.127 | 0.127 |
| Tetraacetic Acid | | | | | | |
| Benzyl Alcohol | 0.400 | 0.400 | 0.400 | 0.006 | 0.006 | 0.006 |
| Methylchloroisothiazolinone, | 0.0005 | 0.0005 | 0.0005 | 0.0005 | 0.0005 | 0.0005 |
| methylisothiazolinone | | | | | | |
| Perfume Booster Accord | 0.250 | 0.300 | 0.350 | 0.350 | 0.500 | 0.500 |
| Panthenyl Etheyl Ether | 0.225 | 0.050 | 0.050 | 0.050 | 0.050 | 0.050 |
| Panthenol | 0.025 | 0.050 | 0.050 | 0.050 | 0.050 | 0.050 |
| Sodium hydroxide | | | 0.0136 | 0.0136 | 0.0136 | 0.0136 |
| Isopropyl Alcohol | | | 0.899 | 0.916 | 0.598 | 0.598 |
| Water/minors | qs | qs | qs | qs | qs | qs |

### Method for Selecting High Odor Value Materials for a Perfume Composition

Methods for selecting high odor value materials for use in perfume composition (e.g., a perfume booster accord composition) are provided herein. In one exemplary embodiment, the method comprises the steps of: (a) identifying a first list of high odor value materials; (b) determining an odor detection threshold value of each of the high odor value materials; (c) determining a human recognition slope factor value of each of the high odor value materials; and (d) selecting the high odor value materials that have both a predetermined odor detection threshold value and a predetermined human recognition slope factor.

The first list of high odor value materials includes all of those known or identifiable by a person of ordinary skill in the art of perfumes and fragrances. The predetermined odor detection threshold may be, for example, less than or equal to about 50 ppb, less than or equal to about 25 ppb, or less than or equal to about 10 ppb. The predetermined human recognition slope factor may be, for example, greater than or equal to about 600, or greater than or equal to about 800.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

What is claimed is:

1. A personal care composition comprising a perfume booster accord,
wherein said perfume booster accord is present in an amount of from about 0.01% to about 10% of said personal care composition,
wherein said perfume booster accord comprises at least two high odor value materials,
wherein said high odor value materials have an odor detection threshold of less than or equal to about 50 ppb,
wherein said high odor value materials have a human recognition slope factor greater than or equal to about 600,
wherein said high odor value materials are present in an amount of from about 0.05% to about 100% of said perfume booster accord.

2. The personal care composition of claim 1, wherein said high odor value materials are present in an amount of from about 0.10% to about 70% of said perfume booster accord.

3. The personal care composition of claim 1, wherein said high odor value materials have an odor detection threshold of less than or equal to about 25 ppb.

4. The personal care composition of claim 1, wherein said high odor value materials have an odor detection threshold of less than or equal to about 10 ppb.

5. The personal care composition of claim 1, wherein said high odor value materials have a human recognition slope factor greater than or equal to about 800.

6. The personal care composition of claim 1, wherein said perfume booster accord is present in an amount of from about 0.05% to about 5% of said personal care composition.

7. The personal care composition of claim 1, wherein said perfume booster accord is present in an amount of from about 0.1% to about 1% of said personal care composition.

8. The personal care composition of claim 1, wherein said perfume booster accord perfume booster accord comprises from about 5 to about 30 high odor value materials.

9. The personal care composition of claim 1, wherein said perfume booster accord perfume booster accord comprises from about 10 to about 20 high odor value materials.

10. The personal care composition of claim 1, wherein said personal care composition is in the form of a composition selected from the group consisting of shampoo, conditioner, antiperspirant, deodorant, hair styling products, cleansers, soaps, bar soap, bodywash, cosmetics, foundations, lotions, creams, ointments, and hydro-alcoholic solutions.

11. The personal care composition of claim 1, wherein said personal care composition is a multi-phase personal care composition.

12. The personal care composition of claim 1, wherein said perfume booster accord is in an encapsulated form.

13. A personal care composition, comprising:
a perfume booster accord comprising at least two high odor value materials selected from the group consisting of 4-(2,6,6-Trimethyl-1-cyclohexenyl)-3-butenone-2; (3aR-(3aalpha,5abeta,9aalpha,9bbeta))-dodecahydro-3a,6,6,9a-tetramethyl naphtha(2,1-b)furan; 2,6-Dimethyl-5-heptenal; 3,7-Dimethyl-1,6-octadien-3-ol; 3-Methyl-2-buten-1-yl acetate; 3,7-Dimethyl-2,6-octadienenitrile; Ethyl-2-methyl-butyrate; 2,4-Dimethylcyclohexene-3-carbaldehyde; Phenyl Acetaldehyde, Indol, ethyl methyl dioxolane acetate; 4-(2,6,6-Trimethyl-1,3-cyclohexadienyl)-3-buten-4-one; Cis 3 Hexenyl Acetate; Lauric Ald, Tricyclo decenyl acetate, Para cresyl methyl ether, 7-acetyl, 1,2,3,4,5,6,7,8-octahydro-1,1,6,7-tetramethyl naphthalene; 3-buten-2-one; 3-methyl-4-(2,6,6-trimethyl-2-cyclohexen-1-yl); Acetic acid (Cyclohexyloxy), 2-propenyl ester; 3-buten-2-one, 4-(2,6,6-trimethyl-2-cyclohexen-1-yl), (E); Decyl Aldehyde, Methyl-3,4-dioxy(cylcoacetonyl) benzene; 2,6-Dimethyl-2,6-octadien-8-ol; ortho tertiary butyl cyclohexanyl acetate; Hexanoic acid,2-propenyl ester; 4-Methoxybenzaldehyde; 3-(3-Isopropylphenyl)butanal; Iso 2-Methoxy-4-(2-propenyl)phenol, Tetra Hydro 3,7-Dimethyl-1,6-octadien-3-ol; Methyl cedrylone; 1-methyl-4-isopropenyl-1-cyclohexene; Methyl phenyl carbonyl acetate; Methyl dihydro jasmonate; Hexahydro-4,7methano-1H-inden-5(or 6)-yl propionate; Benzaldehyde, 3,7-Dimethyl-2,6-octadienal; 3,3-Dimethyl-5-(2,2,3-trimethyl-3-cycloenten-1-yl)-4-penten-2-ol; 2-Methoxy-4-(2-propenyl)phenol; 3,7-dimethyl-6-octen-1-ol; undecalactone; Allyl heptanoate; 1,3-Oxathiane, 2-methyl-4-propyl-, cis-; paradiff; (all-E)-alpha-sinensal 2,6,10-trimethyl-2(E),6(E),9(E), 11-dodecatetraenal; mandarin aldehyde, p-1-menthen-8thiol; Beta gamma hexenol; 4-Methyl-3-decen-5-ol; Ethyl caproate, Ethyl-2-4-decadienoate, 4-Penten-1-one, 1-(5,5-Dimethyl-1-cyclohexen-1-yl)-; 1H-Indene-a-propanal, 2,3-dihydro-1,1-dimethyl-(9CI); Methyl nonyl acetaldehyde; Orange juice Carbonyls; 4 dodecenal; 3-cyclohexene-1-carboxaldehyde, 2,4-dimethyl; 2,6,-nonenol; 2,6-nonadeinal; 2,6-nonadienol; Amyl acetate; 3-P-cumenyl-propionaldehyde 4-(1-methylethyl)-benzenepropanal; 1-(2,6,6-Trimethyl-1,3-cyclohexandienyl)-2-buten-1-one; 6-(Z,3-pentenyl)-tetrahydro-(2H)-pyranone-2; and 3-Methyl-(cis-2-penten-1-yl)-2-cyclopenten-1-one;
wherein said perfume booster accord is present in an amount of from about 0.05% to about 5% of said personal care composition.

14. The personal care composition of claim 13, wherein said perfume booster accord is present in an amount of from about 0.1% to about 1% of said personal care composition.

15. The personal care composition of claim 13, wherein said high odor value materials are not encapsulated.

16. The personal care composition of claim 13, wherein the perfume booster accord comprises at least five of said high odor value materials selected from the group consisting of 4-(2,6,6-Trimethyl-1-cyclohexenyl)-3-butenone-2; (3 aR-(3aalpha,5abeta,9aalpha,9bbeta))-dodecahydro-3a,6,6,9a-tetramethyl naphtha(2,1-b)furan; 2,6-Dimethyl-5-heptenal; 3,7-Dimethyl-1,6-octadien-3-ol; 3-Methyl-2-buten-1-yl acetate; 3,7-Dimethyl-2,6-octadienenitrile; Ethyl-2-methyl-butyrate; 2,4-Dimethylcyclohexene-3-carbaldehyde; Phenyl Acetaldehyde, Indol, ethyl methyl dioxolane acetate; 4-(2,6,6-Trimethyl-1,3-cyclohexadienyl)-3-buten-4-one; Cis 3 Hexenyl Acetate; Lauric Ald, Tricyclo decenyl acetate, Para cresyl methyl ether, 7-acetyl, 1,2,3,4,5,6,7,8-octahydro-1,1,6,7-tetramethyl naphthalene; 3-buten-2-one; 3-methyl-4-(2,6,6-trimethyl-2-cyclohexen-1-yl); Acetic acid (Cyclohexyloxy), 2-propenyl ester; 3-buten-2-one, 4-(2,6,6-trimethyl-2-cyclohexen-1-yl), (E); Decyl Aldehyde, Methyl-3,4-dioxy(cylcoacetonyl) benzene; 2,6-Dimethyl-2,6-octadien-8-ol; ortho tertiary butyl cyclohexanyl acetate; Hexanoic acid,2-propenyl ester; 4-Methoxybenzaldehyde; 3-(3-Isopropylphenyl)butanal; Iso 2-Methoxy-4-(2-propenyl)phenol, Tetra Hydro 3,7-Dimethyl-1,6-octadien-3-ol; Methyl cedrylone; 1-methyl-4-isopropenyl-1-cyclohexene; Methyl phenyl carbonyl acetate; Methyl dihydro jasmonate; Hexahydro-4,7methano-1H-inden-5(or 6)-yl propionate; Benzaldehyde, 3,7-Dimethyl-2,6-octadienal; 3,3-Dimethyl-5-(2,2,3-trimethyl-3-cycloenten-1-yl)-4-penten-2-ol; 2-Methoxy-4-(2-propenyl)phenol; 3,7-dimethyl-6-octen-1-ol; undecalactone; Allyl heptanoate; 1,3-Oxathiane, 2-methyl-4-propyl-, cis-; paradiff; (all-E)-alpha-sinensal 2,6,10-trimethyl-2(E),6(E),9(E), 11-dodecatetraenal; mandarin aldehyde, p-1-menthen-8thiol; Beta gamma hexenol; 4-Methyl-3-decen-5-ol; Ethyl caproate, Ethyl-2-4-decadienoate, 4-Penten-1-one, 1-(5,5-Dimethyl-1-cyclohexen-1-yl)-; 1H-Indene-a-propanal, 2,3-dihydro-1,1-dimethyl-(9CI); Methyl nonyl acetaldehyde; Orange juice Carbonyls; 4 dodecenal; 3-cyclohexene-1-carboxaldehyde, 2,4-dimethyl; 2,6,-nonenol; 2,6-nonadeinal; 2,6-nonadienol; Amyl acetate; 3-P-cumenyl-propionaldehyde 4-(1-methylethyl)-benzenepropanal; 1-(2,6,6-Trimethyl-1,3-cyclohexandienyl)-2-buten-1-one; 6-(Z,3-pentenyl)-tetrahydro-(2H)-pyranone-2; and 3-Methyl-(cis-2-penten-1-yl)-2-cyclopenten-1-one.

17. The personal care composition of claim 13, wherein the perfume booster accord comprises at least ten of said high odor value materials selected from the group consisting of 4-(2,6,6-Trimethyl-1-cyclohexenyl)-3-butenone-2; (3aR-(3aalpha,5abeta,9aalpha,9bbeta))-dodecahydro-3a,6,6,9a-tetramethyl naphtha(2,1-b)furan; 2,6-Dimethyl-5-heptenal; 3,7-Dimethyl-1,6-octadien-3-ol; 3-Methyl-2-buten-1-yl acetate; 3,7-Dimethyl-2,6-octadienenitrile; Ethyl-2-methyl-butyrate; 2,4-Dimethylcyclohexene-3-carbaldehyde; Phenyl Acetaldehyde, Indol, ethyl methyl dioxolane acetate; 4-(2,6,6-Trimethyl-1,3-cyclohexadienyl)-3-buten-4-one; Cis 3 Hexenyl Acetate; Lauric Ald, Tricyclo decenyl acetate, Para cresyl methyl ether, 7-acetyl, 1,2,3,4,5,6,7,8-octahydro-1,1,6,7-tetramethyl naphthalene; 3-buten-2-one; 3-methyl-4-(2,6,6-trimethyl-2-cyclohexen-1-yl); Acetic acid (Cyclohexyloxy), 2-propenyl ester; 3-buten-2-one, 4-(2,6,6-trimethyl-2-cyclohexen-1-yl), (E); Decyl Aldehyde, Methyl-3,4-dioxy(cylcoacetonyl) benzene; 2,6-Dimethyl-2,6-octadien-8-ol; ortho tertiary butyl cyclohexanyl acetate; Hexanoic acid,2-propenyl ester; 4-Methoxybenzaldehyde; 3-(3-Isopropylphenyl)butanal; Iso 2-Methoxy-4-(2-propenyl)phenol, Tetra Hydro 3,7-Dimethyl-1,6-octadien-3-ol; Methyl cedrylone; 1-methyl-4-isopropenyl-1-cyclohexene; Methyl phenyl carbonyl acetate; Methyl dihydro jasmonate; Hexahydro-4,7methano-1H-inden-5(or 6)-yl propionate; Benzaldehyde, 3,7-Dimethyl-2,6-octadienal; 3,3-Dimethyl-5-(2,2,3-trimethyl-3-cycloenten-1-yl)-4-penten-2-ol; 2-Methoxy-4-(2-propenyl)phenol; 3,7-dimethyl-6-octen-1-ol; undecalactone; Allyl heptanoate; 1,3-Oxathiane, 2-methyl-4-propyl-, cis-; paradiff; (all-E)-alpha-sinensal 2,6,10-trimethyl-2(E),6(E),9(E), 1-dodecatetraenal; mandarin aldehyde, p-1-menthen-8thiol; Beta gamma hexenol; 4-Methyl-3-decen-5-ol; Ethyl caproate, Ethyl-2-4-decadienoate, 4-Penten-1-one, 1-(5,5-Dimethyl-1-cyclohexen-1-yl)-; 1H-Indene-a-propanal, 2,3-dihydro-1,1-dimethyl-(9CI); Methyl nonyl acetaldehyde; Orange juice Carbonyls; 4 dodecenal; 3-cyclohexene-1-carboxaldehyde, 2,4-dimethyl; 2,6,-nonenol; 2,6-nonadeinal; 2,6-nonadienol; Amyl acetate; 3-P-cumenyl-propionaldehyde 4-(1-methylethyl)-benzenepropanal; 1-(2,6,6-Trimethyl-1,3-cyclohexandienyl)-2-buten-1-one; 6-(Z,3-pentenyl)-tetrahydro-(2H)-pyranone-2; and 3-Methyl-(cis-2-penten-1-yl)-2-cyclopenten-1-one.

18. The personal care composition of claim 13, wherein the perfume booster accord comprises at least twenty of said high odor value materials selected from the group consisting of 4-(2,6,6-Trimethyl-1-cyclohexenyl)-3-butenone-2; (3aR-(3aalpha,5abeta,9aalpha,9bbeta))-dodecahydro-3a,6,6,9a-tetramethyl naphtha(2,1-b)furan; 2,6-Dimethyl-5-heptenal; 3,7-Dimethyl-1,6-octadien-3-ol; 3-Methyl-2-buten-1-yl acetate; 3,7-Dimethyl-2,6-octadienenitrile; Ethyl-2-methyl-butyrate; 2,4-Dimethylcyclohexene-3-carbaldehyde; Phenyl Acetaldehyde, Indol, ethyl methyl dioxolane acetate; 4-(2,6,6-Trimethyl-1,3-cyclohexadienyl)-3-buten-4-one; Cis 3 Hexenyl Acetate; Lauric Ald, Tricyclo decenyl acetate, Para cresyl methyl ether, 7-acetyl, 1,2,3,4,5,6,7,8-octahydro-1,1,6,7-tetramethyl naphthalene; 3-buten-2-one; 3-methyl-4-(2,6,6-trimethyl-2-cyclohexen-1-yl); Acetic acid (Cyclohexyloxy), 2-propenyl ester; 3-buten-2-one, 4-(2,6,6-trimethyl-2-cyclohexen-1-yl), (E); Decyl Aldehyde, Methyl-3,4-dioxy(cylcoacetonyl) benzene; 2,6-Dimethyl-2,6-octadien-8-ol; ortho tertiary butyl cyclohexanyl acetate; Hexanoic acid,2-propenyl ester; 4-Methoxybenzaldehyde; 3-(3-Isopropylphenyl)butanal; Iso 2-Methoxy-4-(2-propenyl)phenol, Tetra Hydro 3,7-Dimethyl-1,6-octadien-3-ol; Methyl cedrylone; 1-methyl-4-isopropenyl-1-cyclohexene; Methyl phenyl carbonyl acetate; Methyl dihydro jasmonate; Hexahydro-4,7methano-1H-inden-5(or 6)-yl propionate; Benzaldehyde, 3,7-Dimethyl-2,6-octadienal; 3,3-Dimethyl-5-(2,2,3-trimethyl-3-cycloenten-1-yl)-4-penten-2-ol; 2-Methoxy-4-(2-propenyl)phenol; 3,7-dimethyl-6-octen-1-ol; undecalactone; Allyl heptanoate; 1,3-Oxathiane, 2-methyl-4-propyl-, cis-; paradiff; (all-E)-alpha-sinensal 2,6,10-trimethyl-2(E),6(E),9(E), 11-dodecatetraenal; mandarin aldehyde, p-1-menthen-8thiol; Beta gamma hexenol; 4-Methyl-3-decen-5-ol; Ethyl caproate, Ethyl-2-4-decadienoate, 4-Penten-1-one, 1-(5,5-Dimethyl-1-cyclohexen-1-yl)-; 1H-Indene-a-propanal, 2,3-dihydro-1,1-dimethyl-(9CI); Methyl nonyl acetaldehyde; Orange juice Carbonyls; 4 dodecenal; 3-cyclohexene-1-carboxaldehyde, 2,4-dimethyl; 2,6,-nonenol; 2,6-nonadeinal; 2,6-nonadienol; Amyl acetate; 3-P-cumenyl-propionaldehyde 4-(1-methylethyl)-benzenepropanal; 1-(2,6,6-Trimethyl-1,3-cyclohexandienyl)-2-buten-1-one; 6-(Z,3-pentenyl)-tetrahydro-(2H)-pyranone-2; and 3-Methyl-(cis-2-penten-1-yl)-2-cyclopenten-1-one.

19. The personal care composition of claim 13, wherein said perfume booster accord comprises 2,6 nonenol; 2,6, nonadienal; 2,6-nonadienol; (3aR-(3aalpha,5abeta,9aalpha,9bbeta))-dodecahydro-3a,6,6,9a-tetramethyl naphtha(2,1-b)furan; Amyl Acetate; Beta Gamma Hexenol; Cis 3 Hexenyl Acetate; 3-P-cumenyl-propionaldehyde 4-(1-methylethyl)-benzenepropanal; 1-(2,6,6-Trimethyl-1,3-cyclohexandienyl)-2-buten-1-one; Ethyl-2-methyl butyrate; 3-(3-Isopropylphenyl)butanal; 4-Penten-1-one, 1-(5,5-Dimethyl-1-cyclohexen-1-yl)-; 1H-Indene-a-propanal, 2,3-dihydro-1,1-dimethyl-(9CI); 4-(2,6,6-Trimethyl-1-cyclohexenyl)-3-butenone-2; 6-(Z,3-pentenyl)-tetrahydro-(2H)-pyranone-2; 2,6-Dimethyl-5-heptenal; 6,6-Dimethylbicyclo {3.1.1}Hept-2-ene-2-proponal; 3-cyclohexene-1-carboxaldehyde, 2,4-dimethyl; Undecalactone; 4-Methyl-3-decen-5-ol; ortho tertiary butyl cyclohexanyl acetate; 3-Methyl-(cis-2-penten-1-yl)-2-cyclopenten-1-one; and 4-Pentene-2-ol, 3,3-dimethyl-5-(2,2,3-trimethyl-3-cyclopenten-1-yl)-.

20. The personal care composition of claim 13, wherein said perfume booster accord comprises Benzaldehyde; Undeclactone; 4-(2,6,6-Trimethyl-1-cyclohexenyl)-3-butenone-2; Allyl Heptanoate; 1,3-Oxathiane, 2-methyl-4-propyl-, cis-; Paradiff, (all-E)-alpha-sinensal 2,6,10-trimethyl-2(E),6(E),9(E), 11-dodecatetraenal; mandarin aldehyde; 4-dodecenal; p-1-menthen-8 thiol; Beta Gamma Hexenol, Cis 3 Hexenyl Acetate, Orange Juice Carbonyls; ortho tertiary butyl cyclohexanyl acetate; Decyl Aldehyde; 4-Methyl-3-decen-5-ol; and 4-Penten-1-one, 1-(5,5-Dimethyl-1-cyclohexen-1-yl)-.

21. The personal care composition of claim 13, wherein said perfume booster accord comprises Hexanoic acid,2-propenyl ester; 4-Methoxybenzaldehyde; Allyl Heptanoate; Benzaldehyde; Beta Gamma Hexenol; 1,3-Oxathiane, 2-methyl-4-propyl-, cis-; Cis 3 Hexenyl Acetate; Decyl Aldehyde; Ethyl 2′4-decadienoate; Ethyl Caproate; Ethyl-2-methyl Butyrate; 4-Penten-1-one, 1-(5,5-Dimethyl-1-cyclohexen-1-yl)-; p-1-menthen-8thiol; (all-E)-alpha-sinensal 2,6,10-trimethyl-2(E),6(E),9(E), 11-dodecatetraenal; 1H-Indene-a-propanal, 2,3-dihydro-1,1-dimethyl-(9CI); 4-(2,6,6-Trimethyl-1-cyclohexenyl)-3-butenone-2; 3 dodecenal; Methyl Nonyl Acetaldehyde; Orange Juice Carbonyls; Paradiff; undecalactone; 4 dodecenal; 3-cyclohexene-1-carboxaldehyde, 2,4-dimethyl; 4-Methyl-3-decen-5-ol; and ortho tertiary butyl cyclohexanyl acetate.

22. The personal care composition of claim 13, further comprising a perfume solution comprising at least one fragrance material.

23. The personal care composition of claim 22, wherein said perfume solution is present in an amount of from about 0.01% to about 5.0% by weight of said personal care composition.

24. A method for selecting high odor value materials for use in a perfume composition, the method comprising the steps of:
(a) identifying a first list of high odor value materials;
(b) determining an odor detection threshold value of each of said high odor value materials;
(c) determining a human recognition slope factor value of each of said high odor value materials; and
(d) selecting said high odor value materials that have both an odor detection threshold value that is less than or equal to about 50 ppb, and a human recognition slope factor value that is greater than or equal to about 600.

25. The method of claim 24, wherein step (d) includes selecting high odor value materials that have an odor detection threshold value that is less than or equal to about 25 ppb.

26. The method of claim 24, wherein step (d) includes selecting high odor value materials that have an odor detection threshold value that is less than or equal to about 10 ppb.

27. The method of claim 24, wherein step (d) includes selecting high odor value materials that have a human recognition slope factor value that is greater than or equal to about 800.
